(19) United States
(12) Patent Application Publication
(10) Pub. No.: US 2026/0124848 A1
(43) Pub. Date: May 7, 2026

(54) CUSTOMIZABLE INFORMATIONAL CARD FOR HEALTH INFORMATION EXCHANGE WITH LOCAL AND GLOBAL ESTABLISHMENTS FOR SAFER INTERACTIONS

(71) Applicant: Rojin Natalie Ghobadi, Trabuco Canyon, CA (US)

(72) Inventor: Rojin Natalie Ghobadi, Trabuco Canyon, CA (US)

(21) Appl. No.: 19/227,297

(22) Filed: Jun. 3, 2025

Related U.S. Application Data
(60) Provisional application No. 63/663,793, filed on Jun. 25, 2024.

(51) Int. Cl. B42D 25/23; B42D 25/36; G06K 19/077; G16H 10/65

(52) U.S. Cl. CPC B42D 25/23; B42D 25/36; G06K 19/077; G16H 10/65

(57) ABSTRACT

A customizable biodegradable health information card is disclosed. The card allows individuals to personalize the health information displayed, facilitating secure and efficient communication with establishments globally and locally. Unlike existing health information exchange solutions, this invention is tailored to individual needs, supports multiple languages, and is environmentally sustainable due to its biodegradable materials. The card serves as a reusable yet disposable medium, reducing plastic waste while enhancing safety in various establishments such as healthcare facilities, restaurants, and travel hubs. Future iterations of this invention will incorporate optional digital support, including QR codes, NFC chips, and digital wallet storage, for enhanced usability without compromising user privacy.

### FIELD OF THE INVENTION

This invention relates to health communication tools, specifically personalized health information cards that facilitate safe interactions between individuals and establishments. More particularly, it pertains to a card system (biodegradable plastic cards) that allows users to efficiently communicate their medical conditions, dietary restrictions, or other health concerns without relying on digital technology. While incorporating digital enhancements, including NFC and digital wallet integration for seamless tap transactions is an option.

### BACKGROUND OF THE INVENTION

### Description of Related Art

Current solutions for health-related communication include medical alert bracelets, digital health apps, and printed documents. However, these methods present limitations:
1. Medical alert bracelets are often limited in space and information customization.
2. Digital health apps require devices, internet access, and are vulnerable to data privacy risks.
3. Printed documents lack durability and can be lost or damaged easily.

Existing physical cards are generally made from plastic and contribute to environmental pollution. There is a need for a biodegradable yet durable alternative that improves communication while being eco-friendly.

### SUMMARY OF THE INVENTION

The customizable biodegradable health information card addresses these gaps by:
1. Providing customizable information fields, allowing individuals to tailor their health data.
2. Supporting multiple languages, making it accessible for global use.
3. Utilizing sourced biodegradable materials, ensuring environmental sustainability.
4. Ensuring clear, durable print visibility, preventing smudging and wear.
5. Enabling easy transport and sharing, without requiring digital access.
6. Incorporating digital support, such as QR codes, NFC chips, and digital wallet integration, to enhance accessibility over time.

This invention allows individuals to carry a personalized health card that they can present at restaurants, hospitals, airports, and other public spaces to communicate vital health information effectively. The design focuses on printed informational cards as well as digital integration iterations, including the ability to store the card in a digital wallet for tap transactions at compatible establishments

### DETAILED DESCRIPTION OF THE INVENTION

### Structure and Composition

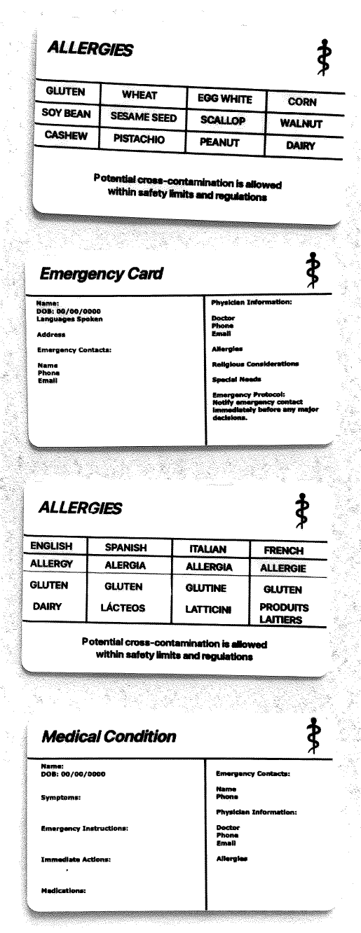

As shown in FIG. 1, the card is wallet-sized and conforms to standard credit card dimensions (approximately 3.375″×2.125″). It may be produced using biodegradable materials.

Manufactured in-house, the card may be produced using biodegradable materials, such as:
7. Recycled paper with waterproof coating for durability.
8. Cornstarch-based biodegradable plastic for extended usability.
9. Soy-based ink printing to ensure non-toxic, smudge-resistant text visibility.

Optional digital features, such as a QR code and NFC chip embedded into the card, allowing establishments to verify the information online without storing sensitive user data. Additionally, the card may be compatible with digital wallets for seamless tap transactions where necessary.

### Method of Use

10. Customization: Users input their personalized health information via an online or physical form.
11. Printing and Distribution: The card is printed on biodegradable material and delivered to the user.
12. Usage: The individual presents the card at establishments for immediate communication of health concerns.
13. Disposal & Sustainability: The card decomposes naturally after its lifespan, minimizing environmental waste.
14. Digital Integration: QR codes, NFC chips, and digital wallet storage may be embedded to enhance the verification process and facilitate tap transactions at participating locations.

### Key Functional Benefits

1. Portable and Wallet-Sized: Easily fits in standard wallets or badge holders.
2. Secure and Private: No digital data storage, reducing hacking risks.
3. Multiple Use and Disposable: Can be used multiple times but degrades safely when discarded.
4. Expandable Digital Support: QR codes, NFC technology, and digital wallet compatibility could be added for enhanced usability.

### Conclusion

This customizable biodegradable health information card offers a novel, sustainable, and secure method of health communication between individuals and establishments worldwide. By integrating customization, multilingual support, and biodegradability, this invention provides an eco-friendly and effective solution for modern health safety needs. Additionally, digital integration, including QR codes, NFC chips, and digital wallet compatibility, will further enhance accessibility, ensuring that this system evolves with technological advancements while maintaining privacy and security.

1. A customizable biodegradable health information card, comprising:
A. A biodegradable substrate material selected from cornstarch-based bioplastic or recycled waterproof paper or sustainable alternative;
B. A customizable information section, configured to receive user-provided health-related data including allergies, dietary restrictions, emergency contacts, or medical conditions;
C. Multilingual support, wherein the health information is presented in two or more languages;
D. A smudge-resistant ink layer applied to the substrate material to ensure readability over time;
E. A compact, portable form factor configured to fit in standard wallets or identification badge holders;
F. A digital interface comprising at least one of: a printed QR code, an embedded NFC chip, or digital wallet integration for enhanced verification and digital interactions.

2. The card of claim 1, wherein the biodegradable substrate material decomposes under standard composting conditions.

3. The card of claim 1, wherein the customizable information section is printed in a standardized format for universal recognition.

4. The card of claim 1, wherein the multilingual support is implemented via a QR code and NFC chip that link to a translation database.

5. The card of claim 1, wherein no personal data is stored electronically, and the digital interface is configured for offline use, with an optional version enabling secure online verification and digital wallet compatibility.
